(19) United States
(12) Patent Application Publication
(10) Pub. No.: US 2018/0091067 A1
(43) Pub. Date: Mar. 29, 2018

(54) PIEZOELECTRIC ELEMENT, PIEZOELECTRIC ACTUATOR, PIEZOELECTRIC MOTOR, ROBOT, ELECTRONIC COMPONENT TRANSPORTING APPARATUS, PRINTER, ULTRASONIC TRANSDUCER, AND METHOD OF MANUFACTURING PIEZOELECTRIC ELEMENT

(71) Applicant: Seiko Epson Corporation, Tokyo (JP)

(72) Inventors: Tomokazu KOBAYASHI, Shiojiri (JP); Takeshi KOKUBUN, Fujimi (JP); Koichi MOROZUMI, Shiojiri (JP)

(21) Appl. No.: 15/718,512

(22) Filed: Sep. 28, 2017

(30) Foreign Application Priority Data
Sep. 29, 2016 (JP) 2016-190782

(51) Int. Cl. H02N 2/10; H01L 41/047; H01L 41/09; H01L 41/332; H01L 41/317; B06B 1/06; A61B 8/00

(52) U.S. Cl. CPC H02N 2/103; H01L 41/047; H01L 41/0913; B25J 9/12; H01L 41/317; B06B 1/0603; A61B 8/4494; H01L 41/332

(57) ABSTRACT

A piezoelectric element includes: a piezoelectric body; and a first electrode which is disposed on the piezoelectric body, and in which in a plan view viewed from a direction where the first electrode and the piezoelectric body are aligned, a region which is a surface of the piezoelectric body on which the first electrode is disposed, located at a vicinity of the first electrode, and within 10 μm from an outer edge of the first electrode has a crystal surface.

### BACKGROUND

### 1. Technical Field

The present invention relates to a piezoelectric element, a piezoelectric actuator, a piezoelectric motor, a robot, an electronic component transporting apparatus, a printer, an ultrasonic transducer, and a method of manufacturing the piezoelectric element.

### 2. Related Art

For example, JP-A-2008-47689 discloses a liquid droplet ejecting apparatus that ejects liquid droplets by vibrating a diaphragm using a piezoelectric element. In addition, as a method of forming the piezoelectric element on the diaphragm, JP-A-2008-47689 discloses a method of forming the piezoelectric element. First, films of a lower electrode, a piezoelectric body, and an upper electrode in order are formed on the diaphragm to obtain a laminate, and thereafter the laminate is patterned by dry etching.

However, when an upper electrode and a piezoelectric body are patterned by dry etching, a surface of the piezoelectric body is damaged by dry etching, and a damaged layer (altered layer such as an amorphous layer and a microcrystalline layer) is formed on the surface of the piezoelectric body. When such a damaged layer is formed, a mechanical strength of a piezoelectric element decreases and failure probability of a device increases.

### SUMMARY

An advantage of some aspects of the invention is to provide a piezoelectric element, a piezoelectric actuator, a piezoelectric motor, a robot, an electronic component transporting apparatus, a printer, an ultrasonic transducer, and a method of manufacturing the piezoelectric element which can reduce the failure probability.

The advantage can be achieved by the following configurations.

A piezoelectric element according to an aspect of the invention includes: a piezoelectric body; and a first electrode which is disposed on the piezoelectric body, and in which in a plan view viewed from a direction where the first electrode and the piezoelectric body are aligned, a region which is a surface of the piezoelectric body on which the first electrode is disposed, located at a vicinity of the first electrode, and within 10 μm from an outer edge of the first electrode has a crystal surface.

With this configuration, it is possible to suppress the occurrence of cracks, burnout, and the like, and the piezoelectric element which may improve the mechanical strength and reduce the failure probability of the apparatus may be obtained.

In the piezoelectric element according to the aspect of the invention, it is preferable that the piezoelectric element includes: a second electrode which is disposed on the surface of the piezoelectric body opposite to the surface on which the first electrode is disposed, and in which in the plan view viewed from the alignment direction, the second electrode has a portion overlapping with the crystal surface.

With this configuration, the occurrence of cracks, burnout, and the like may be more effectively suppressed.

In the piezoelectric element according to the aspect of the invention, it is preferable that in the plan view viewed from the alignment direction, when a portion overlapping with the first electrode of the piezoelectric body is set as a first portion and a portion located at the vicinity of the first electrode is set as a second portion, a length along the alignment direction of the second portion is shorter than a length along the alignment direction of the first portions.

With this configuration, the occurrence of cracks, burnout, and the like may be more effectively suppressed.

In the piezoelectric element according to the aspect of the invention, it is preferable that the piezoelectric body vibrates in a direction intersecting with the alignment direction.

With this configuration, for example, a piezoelectric actuator, a robot, an electronic component transporting apparatus, the printer, and the ultrasonic transducer using the piezoelectric element may be efficiently driven.

A piezoelectric actuator according to an aspect of the invention includes the piezoelectric element according to the aspect of the invention.

With this configuration, the effect of the piezoelectric element according to the aspect of the invention may be enjoyed, and the piezoelectric actuator with high reliability may be obtained.

A piezoelectric motor according to tan aspect of the invention includes the piezoelectric actuator according to the aspect of the invention.

With this configuration, the effect of the piezoelectric actuator (piezoelectric element) according to the aspect of the invention may be enjoyed, and the piezoelectric motor with high reliability may be obtained.

A robot according to an aspect of the invention includes the piezoelectric element according to the aspect of the invention.

With this configuration, the effect of the piezoelectric element according to the aspect of the invention may be enjoyed, and the robot with high reliability may be obtained.

An electronic component transporting apparatus according to an aspect of the invention includes the piezoelectric element according to the aspect of the invention.

With this configuration, the effect of the piezoelectric element according to the aspect of the invention may be enjoyed, and the electronic component transporting apparatus with high reliability may be obtained.

A printer according to an aspect of the invention includes the piezoelectric element according to the aspect of the invention.

With this configuration, the effect of the piezoelectric element according to the aspect of the invention may be enjoyed, and the printer with high reliability may be obtained.

An ultrasonic transducer according to an aspect of the invention includes the piezoelectric element according to the aspect of the invention.

With this configuration, the effect of the piezoelectric element according to the aspect of the invention may be enjoyed, and the ultrasonic transducer with high reliability may be obtained.

A method of manufacturing a piezoelectric element according to an aspect of the invention includes: preparing a piezoelectric body on which a metal film is disposed; removing a portion of the metal film by dry etching or ion milling and patterning the metal film; and light etching a portion of the piezoelectric body which is exposed from the metal film.

With this configuration, it is possible to suppress the occurrence of cracks, burnout, and the like, and the piezoelectric element which may improve the mechanical strength and reduce the failure probability of the apparatus may be obtained.

It is preferable that the method of manufacturing a piezoelectric element according to the aspect of the invention further includes forming a mask on the metal film, which is performed before the process for patterning the metal film, and removing the mask, which is performed after the process for patterning the metal film and before the process for light etching.

With this configuration, it is possible to more accurately pattern the metal film.

It is preferable that the method of manufacturing a piezoelectric element according to the aspect of the invention further includes removing a portion of the piezoelectric body by dry etching or ion milling and patterning the piezoelectric body, which are performed after the process for patterning the metal film and before the process for light etching.

With this configuration, the patterning of the piezoelectric body may be completed before the light etching.

In the method of manufacturing a piezoelectric element according to the aspect of the invention, it is preferable that the piezoelectric body is formed using a solution method in the preparing process.

With this configuration, the piezoelectric body of a thin film may be easily formed.

### BRIEF DESCRIPTION OF THE DRAWINGS

The invention will be described with reference to the accompanying drawings, wherein like numbers reference like elements.

FIG. 1 is a plan view illustrating an entire configuration of a piezoelectric motor according to a first embodiment of the invention.

FIG. 2 is a sectional view taken along line A-A in FIG. 1.

FIG. 3 is a plan view of a vibration unit included in a piezoelectric actuator shown in FIG. 1.

FIG. 4 is a sectional view taken along line B-B in FIG. 3.

FIG. 5 is a sectional view taken along line C-C in FIG. 3.

FIG. 6 is a partially enlarged plan view of a piezoelectric element.

FIG. 7 is a partially enlarged sectional view of a piezoelectric element according to the related art.

FIG. 8 is a partially enlarged sectional view of the piezoelectric element according to the embodiment.

FIG. 9 is a graph illustrating results of durability evaluation.

FIG. 10 is a schematic diagram illustrating driving of the piezoelectric motor illustrated in FIG. 1.

FIG. 11 is a flowchart illustrating a method of manufacturing the piezoelectric element.

FIG. 12 is a sectional view illustrating a method of manufacturing the piezoelectric element.

FIG. 13 is a sectional view illustrating the method of manufacturing the piezoelectric element.

FIG. 14 is a sectional view illustrating the method of manufacturing the piezoelectric element.

FIG. 15 is a sectional view illustrating the method of manufacturing the piezoelectric element.

FIG. 16 is a sectional view illustrating the method of manufacturing the piezoelectric element.

FIG. 17 is a sectional view illustrating the method of manufacturing the piezoelectric element.

FIG. 18 is a sectional view illustrating the method of manufacturing the piezoelectric element.

FIG. 19 is a perspective view illustrating a robot of a second embodiment of the invention.

FIG. 20 is a perspective view illustrating an electronic component transporting apparatus of a third embodiment of the invention.

FIG. 21 is a perspective view illustrating an electronic component holding portion included in the electronic component transporting apparatus illustrated in FIG. 20.

FIG. 22 is a schematic diagram illustrating an entire configuration of a printer according to a fourth embodiment of the invention.

FIG. 23 is a sectional view of a head included in the printer illustrated in FIG. 22.

FIG. 24 is a sectional view of a piezoelectric element included in the head illustrated in FIG. 23.

FIG. 25 is a schematic diagram illustrating an entire configuration of an ultrasonic transducer according to a fifth embodiment of the invention.

FIG. 26 is a plan view of an element chip included in the ultrasonic transducer illustrated in FIG. 25.

FIG. 27 is a sectional view of a piezoelectric element included in the element chip illustrated in FIG. 26.

### DESCRIPTION OF EXEMPLARY EMBODIMENTS

Hereinafter, a piezoelectric element, a piezoelectric actuator, a piezoelectric motor, a robot, an electronic component transporting apparatus, and a printer, an ultrasonic transducer, and a method of manufacturing the piezoelectric element of the invention will be described in detail based on appropriate embodiments illustrated in the attached drawings.

### First Embodiment

First, the piezoelectric motor according to a first embodiment of the invention will be described.

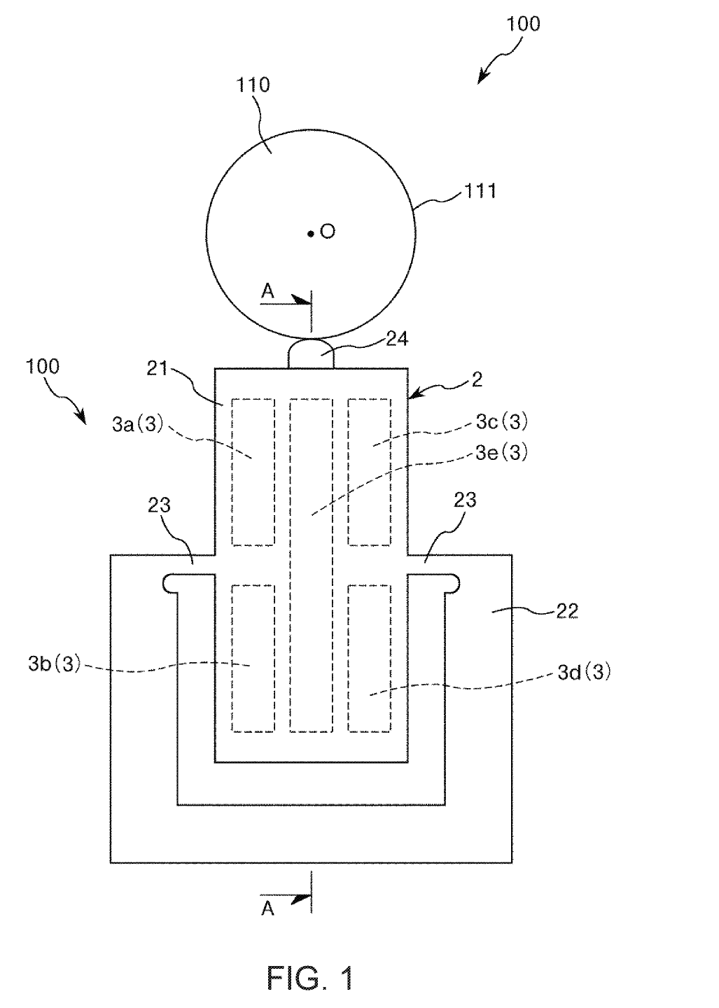

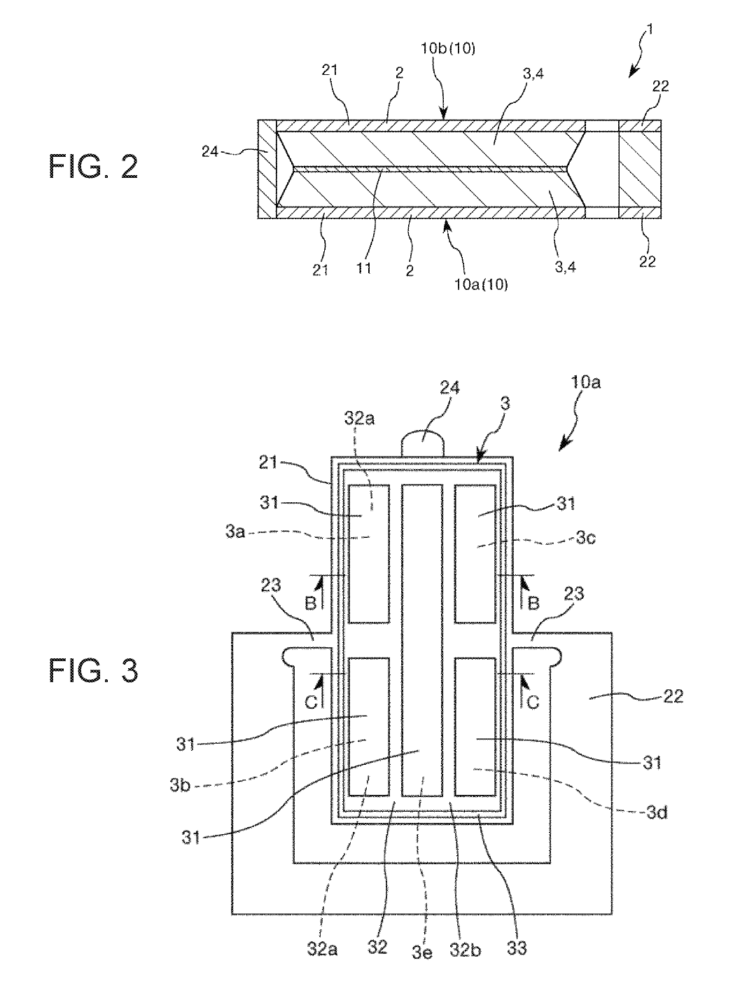

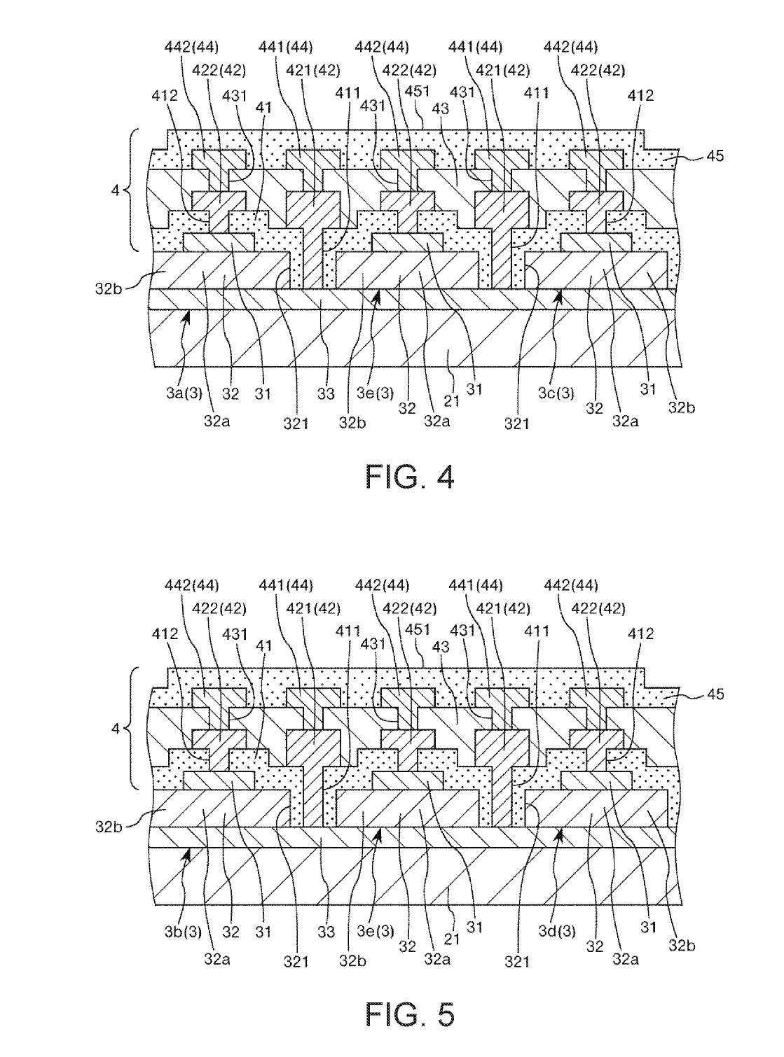

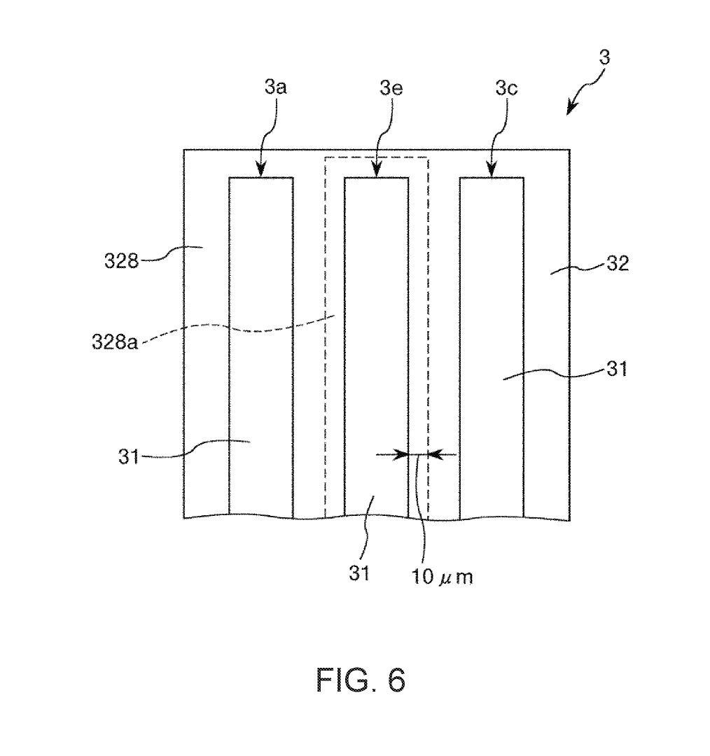

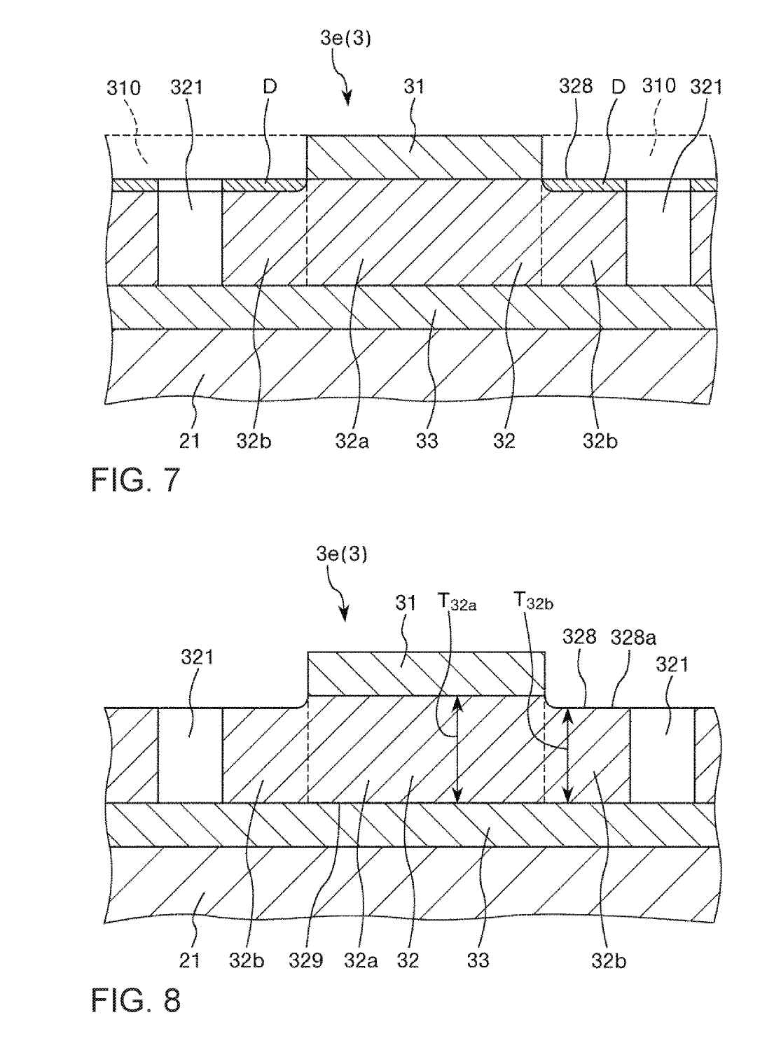

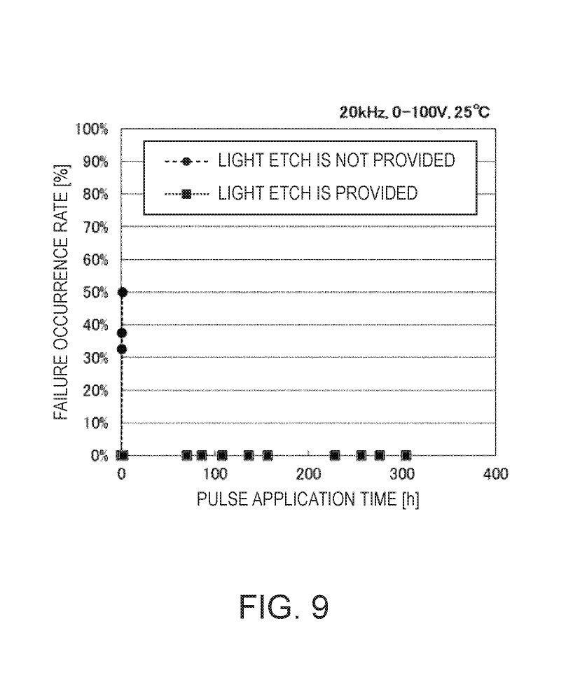

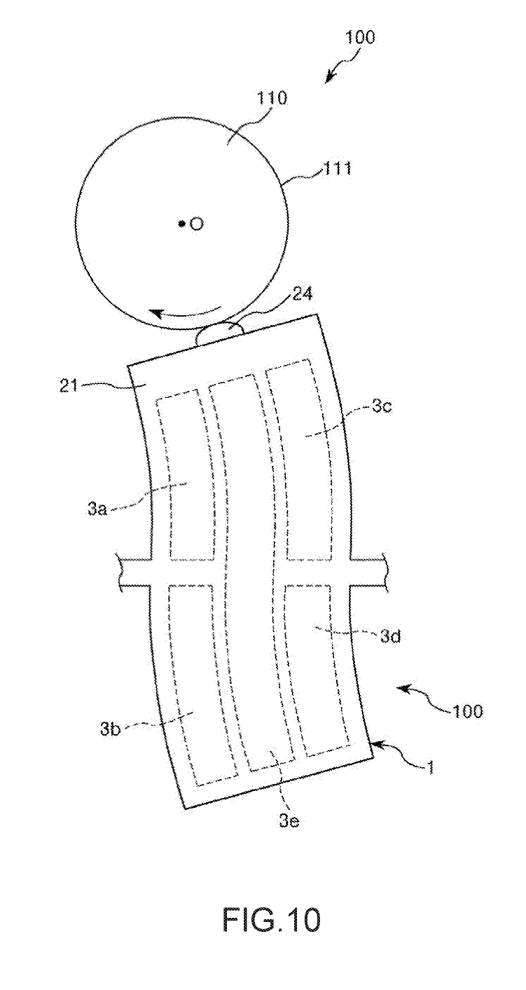

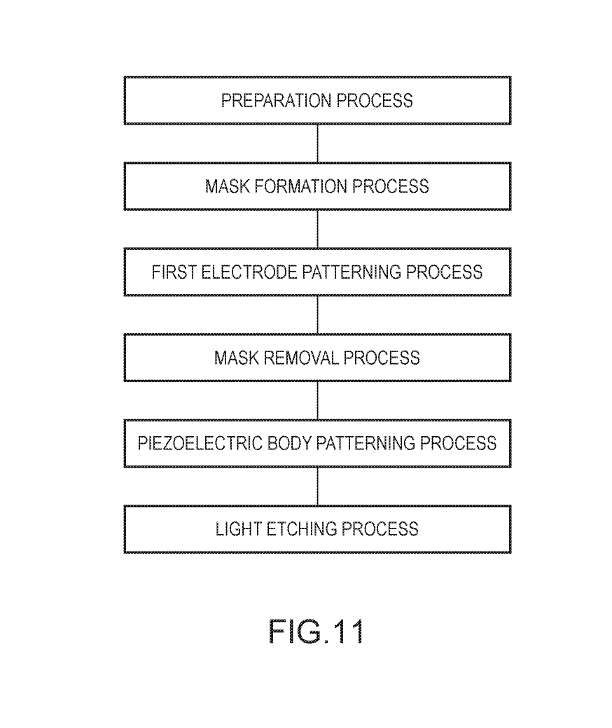

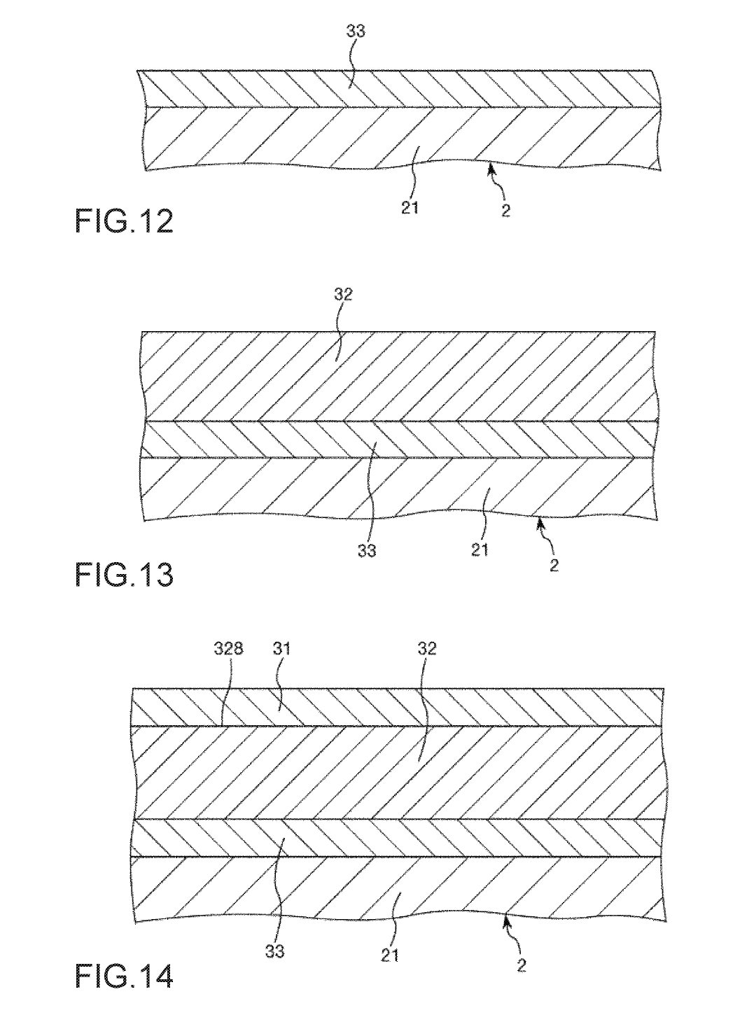

FIG. 1 is a plan view illustrating an entire configuration of a piezoelectric motor according to a first embodiment of the invention. FIG. 2 is a sectional view taken along line A-A in FIG. 1. FIG. 3 is a plan view of a vibration unit included in a piezoelectric actuator shown in FIG. 1. FIG. 4 is a sectional view taken along line B-B in FIG. 3. FIG. 5 is a sectional view taken along line C-C in FIG. 3. FIG. 6 is a partially enlarged plan view of a piezoelectric element. FIG. 7 is a partially enlarged sectional view of a piezoelectric element according to the related art. FIG. 8 is a partially enlarged sectional view of the piezoelectric element according to the embodiment. FIG. 9 is a graph illustrating results of durability evaluation. FIG. 10 is a schematic diagram illustrating driving of the piezoelectric motor illustrated in FIG. 1. FIG. 11 is a flowchart illustrating a method of manufacturing the piezoelectric element. FIGS. 12 to 18 are sectional views illustrating the method of manufacturing the piezoelectric element, respectively. For the convenience of the description, FIG. 2 illustrates the piezoelectric element and a wiring layer in a simplified manner, and the illustration of the wiring layer is omitted in FIG. 3. In addition, hereinafter, for the convenience of the description, the lower side of the drawing paper in FIG. 1 is referred to as “upper”, and the upper side of the drawing paper in FIG. 1 is referred to as “lower”. In addition, the rotor side of the piezoelectric actuator is referred to as the “tip end side”, and the side opposite to the rotor is referred to as the “base end side”.

A piezoelectric motor 100 (ultrasonic motor) illustrated in FIG. 1 has a rotor 110 as a driven portion (driven unit) rotatable around a rotating shaft O and a piezoelectric actuator 1 abutting an outer peripheral surface 111 of the rotor 110. In addition, the piezoelectric actuator 1 is provided with a piezoelectric element 3. In addition, the piezoelectric actuator 1 is biased towards the rotor 110 by a biasing member (not illustrated), and is in contact with the outer peripheral surface 111 with an appropriate frictional force. In such a piezoelectric motor 100, vibration of the piezoelectric element 3 causes the piezoelectric actuator 1 to vibrate, and the vibration of the piezoelectric actuator 1 is transmitted to the rotor 110 so that the rotor 110 rotates around the rotating shaft O.

The configuration of the piezoelectric motor 100 is not limited to the configuration of FIG. 1. For example, a plurality of the piezoelectric actuators 1 may be aligned along a circumferential direction of the rotor 110, and the rotor 110 may be rotated by driving the plurality of the piezoelectric actuators 1. According to such a configuration, the piezoelectric motor 100 can exhibit a larger driving force (torque). In addition, the rotor 110 that rotates and moves as the driven portion is used in the embodiment, but the rotor that linearly moves as the driven portion may be used.

As illustrated in FIG. 2, the piezoelectric actuator 1 has two vibration units 10 (10a and 10b), and these two vibration units 10a and 10b are joined to each other with an insulating adhesive 11 interposed therebetween. However, the number of the vibration units 10 is not particularly limited, and may be one or three or more. In addition, a method of joining the two vibration units 10a and 10b is not limited to joining using the adhesive 11, but may be joined by metal joining, for example.

Next, the vibration units 10a and 10b will be described, since these two vibration units 10a and 10b are similar in configuration to each other, hereinafter, the vibration unit 10a will be described as a representative and the description of the vibration unit 10b is not repeated.

As illustrated in FIG. 3, the vibration unit 10a mainly has a substrate 2 and the piezoelectric element 3 provided on the substrate 2. In addition, the substrate 2 has a vibration portion 21, a support portion 22 supporting the vibration portion 21, and a connection portion 23 connecting the vibration portion 21 and the support portion 22. The piezoelectric element 3 is provided in the vibration portion 21, and when the piezoelectric element 3 is vibrated, the vibration portion 21 can be vibrated in a surface thereof. In addition, a protruding contact portion 24 is provided at a tip end portion of the vibration portion 21, and the tip end of the contact portion 24 is in contact with the outer peripheral surface 111 of the rotor 110 (refer to FIG. 1). The configuration material of the contact portion 24 is not particularly limited, and various ceramics such as alumina, zirconia, silicon nitride and the like can be used, for example. As a result, the hard contact portion 24 is obtained, and wear of the contact portion 24 can be reduced.

In the embodiment, the vibration portion 21 has a substantially rectangular shape (longitudinal shape), the support portion 22 forms a U shape surrounding the base end side of the vibration portion 21, and the connection portion 23 is provided so as to support the vibration portion 21 at both ends thereof on both sides in the width direction of the vibration portion 21, but the shape and arrangement of each part is not particularly limited as long as each part can exhibit functions thereof.

The substrate 2 is not particularly limited, and a silicon substrate can be used, for example. The silicon substrate is used as the substrate 2, so that excellent processing accuracy (dimensional accuracy) can be exhibited. In addition, the semiconductor process can be used for manufacturing the piezoelectric actuator 1, and the piezoelectric actuator 1 can be manufactured efficiently. Although not illustrated, an insulating layer is provided on the surface (surface on the side of the piezoelectric element 3) of the substrate 2. The insulating layer is not particularly limited, and the insulating layer can be configured to include a laminate of, for example, a silicon oxide layer provided on the substrate 2 and a zirconium oxide layer provided on the silicon oxide layer.

As illustrated in FIG. 3, the piezoelectric element 3 includes five piezoelectric elements 3a, 3b, 3c, 3d, and 3e. The piezoelectric element 3e is disposed along the longitudinal direction of the vibration portion 21 at the center portion in the width direction of the vibration portion 21. The piezoelectric elements 3a and 3b are disposed along the longitudinal direction of the vibration portion 21 on one side in the width direction of the vibration portion 21 with respect to the piezoelectric element 3e, and the piezoelectric elements 3c and 3d are disposed along the longitudinal direction of the vibration portion 21 on the other side. In FIG. 3, for the convenience of the description, an illustration of a wiring layer 4 disposed on the piezoelectric element 3 is omitted.

In addition, as illustrated in FIGS. 4 and 5, the five piezoelectric elements 3a, 3b, 3c, 3d, and 3e respectively have a piezoelectric body 32, a first electrode 31 provided on a top surface of the piezoelectric body 32, and a second electrode 33 on a bottom surface of the piezoelectric body 32.

The second electrode 33 is provided on the vibration portion 21. The second electrode 33 is a common electrode commonly provided for the piezoelectric elements 3a, 3b, 3c, 3d, and 3e. Such a second electrode 33 can be configured to include a lamination, for example, of a first titanium (Ti) layer provided on the vibration portion 21, an iridium (Ir) layer provided on the first titanium (Ti) layer, a platinum (Pt) layer provided on the iridium (Ir) layer, and a second titanium (Ti) layer provided on the platinum (Pt) layer. However, the configuration of the second electrode 33 is not limited to the above configuration and may be, for example, a metal material such as titanium (Ti), platinum (Pt), tantalum (Ta), iridium (Ir), strontium (Sr), indium (In), tin (Sn), gold (Au), aluminum (Al), iron (Fe), chromium (Cr), nickel (Ni), copper (Cu), a metal layer made of an alloy material or the like including these metal materials, or a mixture or a laminate of two or more of these.

The piezoelectric body 32 is provided on the second electrode 33 and has a film shape. In addition, the piezoelectric body 32 vibrates (expands and contracts) in the direction along the longitudinal direction of the vibration portion 21 by applying an electric field in the direction along the thickness direction thereof (direction intersecting with the direction in which the first electrode 31 and the piezoelectric body 32 are aligned). As a result, the piezoelectric actuator 1 can be efficiently vibrated in the plane as described later, and furthermore, the rotor 110 can be rotated efficiently.

The thickness of the piezoelectric body 32 is not particularly limited, and the thickness is preferably 50 nm or more and 20 μm or less, more preferably 0.5 μm or more and 7 μm or less, for example. Accordingly, it can be said that the piezoelectric element 3 is the thin film piezoelectric element. When the thickness of the piezoelectric body 32 is smaller than 50 nm, the piezoelectric breakdown is likely to occur, so that the driving voltage cannot be increased and the output of the piezoelectric actuator 1 may be reduced accordingly in some cases. On the other hand, when the thickness of the piezoelectric body 32 is larger than 20 the possibility of cracking in the piezoelectric body 32 increases, which may increase the driving voltage in some cases.

In addition, the piezoelectric body 32 has an active portion 32a and a nonactive portion 32b. The active portion 32a is the portion interposed between the first electrode 31 and the second electrode 33, and actively drives (vibrates) by applying a driving voltage between the first electrode 31 and the second electrode 33. On the other hand, the nonactive portion 32b is the portion not interposed between the first electrode 31 and the second electrode 33 (that is, portion other than the active portion 32a), and is located at the vicinity of the active portion 32a.

In addition, a contact hole 321 penetrating to the second electrode 33 is provided in the nonactive portion 32b. A plurality of the contact holes 321 are provided at the vicinity of the first electrode 31.

The constituent material of the piezoelectric body is not particularly limited, and for example, a piezoelectric material of perovskite type oxide (oxide having a perovskite type crystal structure) can be used. Examples of such a piezoelectric material include lead zirconate titanate (PZT), lead zirconate titanate niobate (PZTN), barium titanate, potassium niobate, lithium niobate, lithium tantalate, and the like.

The first electrode 31 is provided on the piezoelectric body 32. The first electrode 31 is an individual electrode provided individually for each of the piezoelectric elements 3a, 3b, 3c, 3d, and 3e. The first electrode 31 may be, for example, a metal material such as titanium (Ti), platinum (Pt), tantalum (Ta), iridium (Ir), strontium (Sr), indium (In), tin (Sn), gold (Au), aluminum (Al), iron (Fe), chromium (Cr), nickel (Ni), copper (Cu), a metal layer made of an alloy material or the like including these metal materials, or a mixture or a laminate of two or more of these.

Hereinbefore, the configuration of the piezoelectric element 3 has briefly described. Next, the configuration of the piezoelectric element 3 will be described in more detail. Hereinafter, for the convenience of the description, the piezoelectric element 3e and its surrounding configuration will be described as a representative, and the piezoelectric elements 3a, 3b, 3c, 3d and surroundings thereof have the same configuration.

As described above, the piezoelectric element 3e has the piezoelectric body 32 and the first electrode 31 disposed in the piezoelectric body 32. As illustrated in FIG. 6, in a plan view viewed from a direction in which the first electrode 31 and the piezoelectric body 32 are aligned (that is, a plan view viewed from the thickness direction of the film-shape piezoelectric body 32. hereinafter, simply referred to as “plan view”), a region 328a which is a top surface 328 of the piezoelectric body 32 (that is, the surface on the side where the first electrode 31 is disposed), located at a vicinity of the first electrode 31, and within 10 μm from an outer edge of the first electrode 31 has a crystal surface. As a result, as described later, occurrence of cracks, burnout and the like can be suppressed, and accordingly the piezoelectric element 3 which can improve the mechanical strength and reduce the failure probability is obtained.

To explain this fact in more detail, as will be described in the method of manufacturing the piezoelectric element 3 to be described later, the piezoelectric element 3 is obtained by forming the second electrode 33, the piezoelectric body 32, and the first electrode 31 in order on the vibration portion 21 in a solid state, and thereafter patterning the first electrode 31 by dry etching (splitting for the piezoelectric elements 3a, 3b, 3c, 3d, and 3e), and subsequently patterning the piezoelectric body 32 by dry etching (forming contact hole 321). In such a manufacturing process, when the first electrode 31 is patterned by dry etching, the top surface 328 of the piezoelectric body 32 is slightly etched, and the top surface 328 of the piezoelectric body 32 is damaged by the plasma. Therefore, as illustrated in FIG. 7, a damaged layer D is formed in the region overlapping with the portion 310 (that is, nonactive portion 32b) which removed the first electrode 31 of the top surface 328. The damaged layer D is, for example, an altered layer such as an amorphous layer, a microcrystalline layer, and the like, and crystals are collapsed (disturbed) with respect to a normal portion of the piezoelectric body 32 which is not subjected to plasma damage. In the related art, since such damaged layer D remained, cracks, burnout, and the like occurred in the portion in some cases.

Therefore, in the embodiment, the damaged layer D is removed and the top surface 328 of the nonactive portion 32b of the piezoelectric body 32 is configured to include the crystal surface (surface on which the crystal of the piezoelectric body 32 is normally maintained) as illustrated in FIG. 8. In other words, the crystal surface is exposed at the top surface 328 of the nonactive portion 32b of the piezoelectric body 32. Therefore, it is difficult for cracks, burnouts, and the like to occur as described above with respect to the configuration in the related art as described above, and accordingly the mechanical strength of the piezoelectric element 3 is increased and the failure probability can be reduced. If the region 328a of the top surface 328, where is located at the vicinity of the first electrode 31 and within at least 10 μm from the outer edge of the first electrode 31 is configured to include the crystal surface, the above effects can be fully exhibited. In the embodiment, an entire area of the top surface 328, where is located at the vicinity of the first electrode 31 (that is, entire area of the top surface 328 of the nonactive portion 32b) is configured to include the crystal surface. As a result, since all of the damaged layer D can be substantially removed from the top surface 328 of the piezoelectric body 32, it is possible to more effectively suppress occurrence of cracks, burnout, and the like. “Within 10 μm from the outer edge of the first electrode 31” is the distance (length) in plan view viewed from the direction in which the first electrode 31 and the piezoelectric body 32 are aligned.

When the durability evaluation of the piezoelectric actuator was performed between a case where the light etching was performed and a case where the light etching was not performed, as illustrated in FIG. 9, in the piezoelectric actuator which was not subjected to the light etching, half thereof was failed in approximately 2 hours, whereas in the piezoelectric actuator which was subjected to the light etching, failure did not occur even after 300 hours had elapsed. In the evaluation, a sinusoidal wave of 0 to 100 V was used as a driving voltage, and the number of evaluations was 40 (N=40).

The method of removing the damaged layer D is not particularly limited, but light etching using dry etching is preferable. The light etching is, for example, an etching having a low etching rate with respect to dry etching at the time of patterning the first electrode 31. In other words, the dry etching at the time of patterning the first electrode 31 increases the etching rate with emphasis on improving the throughput, whereas the light etching at the time of removing the damaged layer D decreases the etching rate with emphasis on reducing the charging damage over the processing rate. For light etching, for example, light etching can be easily performed by changing the type of gas, the gas ratio, the pressure in the chamber, RF power to be supplied into the chamber for the dry etching at the time of patterning the first electrode 31. By using such light etching, the damaged layer D can be removed while reducing damage to the piezoelectric body 32 (that is, while suppressing the formation of a new damaged layer D).

Although not particularly limited, when the etching rate of dry etching with respect to the piezoelectric body 32 at the time of patterning the first electrode 31 is set as A nm/min, and the etching rate of the light etch with respect to the piezoelectric body 32 at the time of removing the damaged layer D is set as B nm/min, A/B is preferably 10 or more and 50 or less, and more preferably 30 or more and 40 or less. By satisfying such conditions, it is possible to more effectively remove the damaged layer D while reducing damage to the piezoelectric body 32. In addition, it is possible to prevent excessive elongation of the processing time of the light etching.

In addition, as illustrated in FIG. 8, the thickness T_{32b} of the nonactive portion 32b is thinner than the thickness T₃₂ₐ of the active portion 32a. That is, in a plan view, when the portion (active portion 32a) overlapping with the first electrode 31 of the piezoelectric body 32 is set as the first portion, and the portion (nonactive portion 32b) located at the vicinity of the first electrode 31 is set as the second portion, the thickness T_{32b} (length along the direction in which the first electrode 31 and the piezoelectric body 32 are aligned) of the second portion (nonactive portion 32b) is thinner than the thickness T₃₂ₐ (length along the direction in which the first electrode 31 and the piezoelectric body 32 are aligned) of the first portion (active portion 32a). The top surface 328 of the nonactive portion 32b is located below the top surface 328 of the active portion 32a (on the side of the second electrode 33). With such a configuration, the damaged layer D can be more reliably removed. Therefore, it is possible to more effectively suppress the occurrence of cracks, burnout, and the like. In addition, it is possible to easily confirm that the damaged layer D has been removed. Although the thickness is varies depending on the conditions of dry etching at the time of patterning the first electrode 31, the thickness of the damaged layer D is approximately 1 nm or more and approximately 10 nm or less. Therefore, for example, the top surface 328 of the nonactive portion 32b is positioned 10 nm or more below the top surface 328 of the active portion 32a (second electrode 33 side), so that more reliably, the damaged layer D is removed and the top surface 328 of the nonactive portion 32b can be configured to include the surface of the crystal layer.

Here, as described above, in the embodiment, the second electrode 33 is a common electrode of the piezoelectric elements 3a, 3b, 3c, 3d, and 3e. Therefore, as illustrated in FIG. 8, the piezoelectric element 3 has the second electrode 33 disposed on the bottom surface 329 of the piezoelectric body 32 (that is, surface opposite to the surface on which the first electrode 31 is disposed), and in plan view, it can be said that the second electrode 33 has the portion overlapping with the region 328a (crystal surface) of the top surface 328. With such a configuration, when a driving voltage is applied between the first electrode 31 and the second electrode 33, an electric field is generated at the vicinity of the first electrode 31, that is, around the nonactive portion 32b. Therefore, in the configuration having the damaged layer D as in the related art, the electric field is generated up to the vicinity of the damaged layer D, and the possibility of cracks, burnout, and the like is further increased. Accordingly, in a case of the configuration in which the second electrode 33 has the portion overlapping with the region 328a of the top surface 328, it can be said that the occurrence of cracks, burnout, and the like can be more effectively suppressed.

Hereinbefore, the piezoelectric element 3 is described. As illustrated in FIGS. 4 and 5, a first insulating layer 41 is provided on the top surface (surface on the side of the first electrode 31) of such a piezoelectric element 3. The first insulating layer 41 is provided so as to cover the first electrode 31 and the piezoelectric body 32. Such a constituent material of the first insulating layer 41 is not particularly limited, and examples thereof include inorganic materials such as aluminum oxide (AlOx), tantalum oxide (TaOx), hafnium oxide (HfOx), or the like.

In addition, the first insulating layer 41 is provided with contact holes 411 and 412. The contact hole 411 is provided so as to penetrate the contact hole 321 to the second electrode 33. On the other hand, the contact hole 412 is provided so as to penetrate to the top surface of the first electrode 31.

On the top surface of the first insulating layer 41, a first conductive layer 42 is provided. The first conductive layer 42 may be configured to include, for example, a titanium tungsten (TiW) layer and a copper (Cu) layer provided on the titanium tungsten (TiW) layer. For example, the first conductive layer 42 is not limited to this configuration, and may further have a conductive adhesion layer provided between the titanium tungsten (TiW) layer and the first insulating layer 41.

In addition, the first conductive layer 42 has wiring layers 421 and 422. The wiring layer 421 is electrically connected to the second electrode 33 via the contact hole 411. On the other hand, the wiring layer 422 is electrically connected to the first electrode 31 via the contact hole 412.

On the top surface of the first conductive layer 42, a second insulating layer 43 is provided. The second insulating layer 43 has, for example, photosensitivity. Therefore, the second insulating layer 43 can be patterned by exposure, development, and baking (heat treatment) without using etching. The configuration material of the second insulating layer 43 is not particularly limited, and examples thereof include an epoxy resin, an acrylic resin, or the like.

In addition, the second insulating layer 43 is provided with a contact hole 431. The contact hole 431 is provided so as to penetrate to the top surface of the first conductive layer 42.

On the top surface of such a second insulating layer 43, a second conductive layer 44 is provided. In addition, the second conductive layer 44 has wiring layers 441 and 442. The wiring layer 441 is electrically connected to the wiring layer 421 via the contact hole 431. On the other hand, the wiring layer 442 is electrically connected to the wiring layer 422 via the contact hole 431. The configuration material of the second conductive layer 44 is not particularly limited, and may be the same as, for example, that of the first conductive layer 42 described above. Although not illustrated, the piezoelectric actuator 1 is electrically connected to the driving circuit via the second conductive layer 44 (wiring layers 441 and 442), and thus a driving voltage is applied to the piezoelectric element 3 via the second conductive layer 44.

On the top surface of the second conductive layer 44, a third insulating layer 45 is provided. The surface of the third insulating layer 45 is a bonding surface 451. Such a constituent material of the third insulating layer 45 is not particularly limited, and for example, the same material as that of the second insulating layer 43 can be used.

The wiring layer 4 is configured to include the first insulating layer 41, the first conductive layer 42, the second insulating layer 43, the second conductive layer 44, and the third insulating layer 45 as described above.

Hereinbefore, the piezoelectric actuator 1 is described. Such a piezoelectric actuator 1 can be driven, for example, as follows. However, the method of driving the piezoelectric actuator 1 is not limited to the following method. For example, when a drive signal (alternating voltage) of a predetermined frequency is applied to each of the piezoelectric elements 3a, 3b, 3c, 3d, and 3e, so that the phase difference between the piezoelectric elements 3a, 3d and the piezoelectric elements 3b, 3c is 180°, and the phase difference between the piezoelectric elements 3a, 3d and the piezoelectric element 3e is 30°, as illustrated in FIG. 10, each of the piezoelectric elements 3a, 3b, 3c, 3d, and 3e expands and contracts so that the vibration portion 21 bends and deforms in an S-shape in the in-plane direction (expands and contracts in the longitudinal direction and bends and deforms in the width direction afterward), and the tip end of the contact portion 24 elliptically moves. As a result, the rotor 110 rotates in the direction of the arrow around the rotating shaft O thereof. When the drive signal is applied to the piezoelectric element 3e so that the phase difference between the piezoelectric elements 3a and 3d is 210°, the rotor 110 can be rotated in the reverse direction.

Hereinbefore, the piezoelectric element 3, the piezoelectric actuator 1 provided with the piezoelectric element 3, and the piezoelectric motor 100 provided with the piezoelectric actuator 1 have been described in detail. Since the piezoelectric actuator 1 and the piezoelectric motor 100 each have the piezoelectric element 3, and it is possible to enjoy the effect of the piezoelectric element 3 described above, excellent mechanical strength, low failure probability, and excellent reliability can be exhibited.

Next, the method of manufacturing the piezoelectric element 3 will be described. As illustrated in FIG. 11, the method of manufacturing the piezoelectric element 3 mainly includes a preparation process for preparing the piezoelectric body 32 having the top surface 328 as a metal film arrangement surface and the first electrode 31 as a metal film disposed on the top surface 328, a first electrode patterning process of removing a portion of the first electrode 31 by dry etching or ion milling and patterning the first electrode 31, and a light etching process for light etching a portion of the piezoelectric body 32 which is exposed from the first electrode 31. Furthermore, the method of manufacturing the piezoelectric element 3 includes a mask formation process which is performed before the first electrode patterning process to form a mask M1 on the first electrode 31, a mask removal process which is performed after the first electrode patterning process and before the light etching process to remove the mask M1, and a piezoelectric body patterning process which is performed after the first electrode patterning process and before the light etching process to remove a portion of the piezoelectric body 32 by dry etching or ion milling, and to pattern the piezoelectric body. That is, the method of manufacturing the piezoelectric element 3 includes the preparation process, the mask formation process, the first electrode patterning process, the mask removal process, the piezoelectric body patterning process, and the light etching process in order from the start. Hereinafter, the method of manufacturing the piezoelectric element 3 will be described in detail.

### Preparation Process

First, as illustrated in FIG. 12, the substrate 2 serving as the silicon substrate is prepared and the second electrode 33 is formed on the vibration portion 21. The second electrode 33 can be formed, for example, by film formation and patterning (patterning by photolithography and etching) by a sputtering method, a CVD method, a vacuum evaporation method or the like.

Next, as illustrated in FIG. 13, the piezoelectric body 32 is formed on the second electrode 33. The piezoelectric body 32 is formed, for example, by repeating formation of a precursor layer by solution method (liquid phase method) and crystallization of the precursor layer. The liquid phase method is a method of forming a thin film material using a raw material liquid containing constituent materials of a thin film (piezoelectric body 32), specifically, a sol-gel method, a Metal Organic Deposition (MOD) method. The crystallization is performed in an oxygen atmosphere, for example, by heat treatment at 700° C. to 800° C. Specifically, in the preparation process of the embodiment, the piezoelectric body 32 is formed by using the MOD method. As a result, the piezoelectric body 32 of the thin film can be easily formed.

Next, as illustrated in FIG. 14, the first electrode 31 is formed on the top surface 328 (metal film arrangement surface) of the piezoelectric body 32 in a solid state. The first electrode 31 can be formed, for example, in the same manner as the second electrode 33. In the process, the first electrode 31 is formed over the entire top surface 328 of the piezoelectric body 32.

### Mask Formation Process

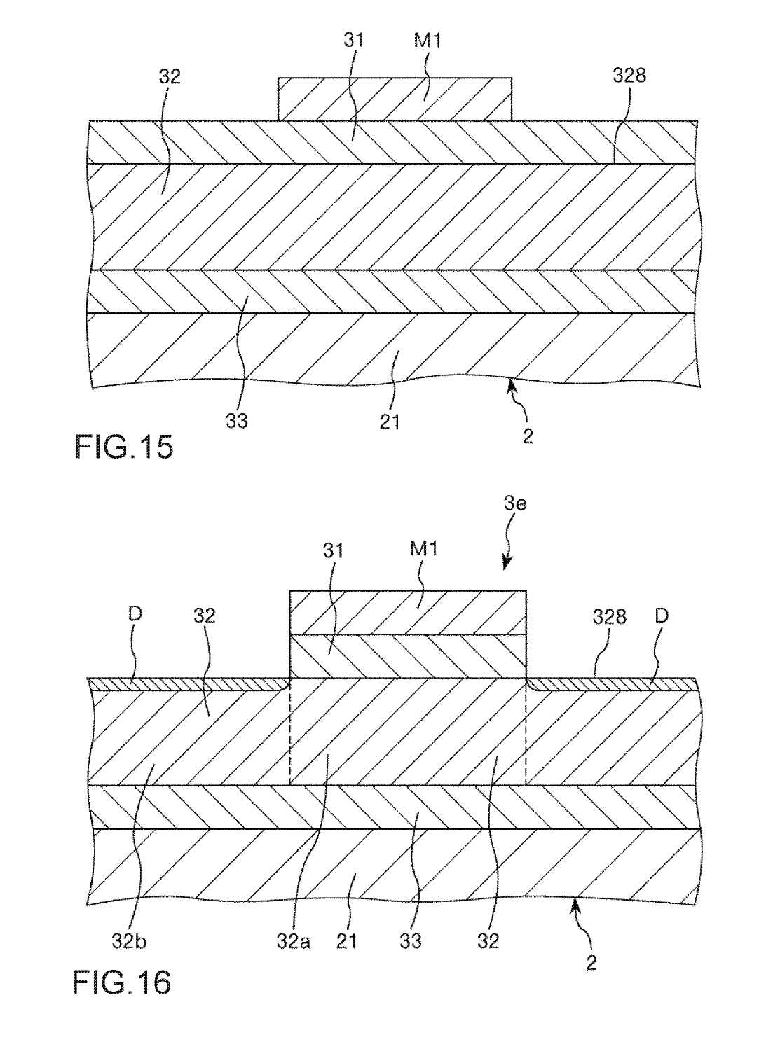

Next, as illustrated in FIG. 15, the mask M1 having an opening corresponding to the division pattern of the first electrode 31 is formed on the first electrode 31. The mask M1 can be formed, for example, by film formation and patterning (patterning by photolithography and etching) by a sputtering method, a CVD method, a vacuum evaporation method, a spin coating method, or the like. The mask M1 is formed on the first electrode 31 in this manner, so that the first electrode 31 can be patterned accurately in the next first electrode patterning process.

### First Electrode Patterning Process

Next, the first electrode 31 is dry-etched via the mask M1, and the first electrode 31 is patterned. As a result, as illustrated in FIG. 16, the active portion 32a and the nonactive portion 32b are formed in the piezoelectric body 32, and the piezoelectric elements 3a, 3b, 3c, 3d, and 3e are obtained (herein, FIG. 16 illustrates only the piezoelectric element 3e). During the dry etching of the process, the top surface 328 of the nonactive portion 32b is over-etched and damaged, and the damaged layer D is formed on the top surface 328 of the nonactive portion 32b. The damaged layer D is an altered layer such as an amorphous layer, a microcrystalline layer, for example, and the crystal collapses with respect to the normal portion where the piezoelectric body 32 is not plasma-damaged.

### Mask Removal Process

Next, the mask M1 is removed by asking treatment. By removing the mask M1 at this stage, it is possible to prevent the mask M1 from obstructing the subsequent piezoelectric body patterning process.

### Piezoelectric Body Patterning Process

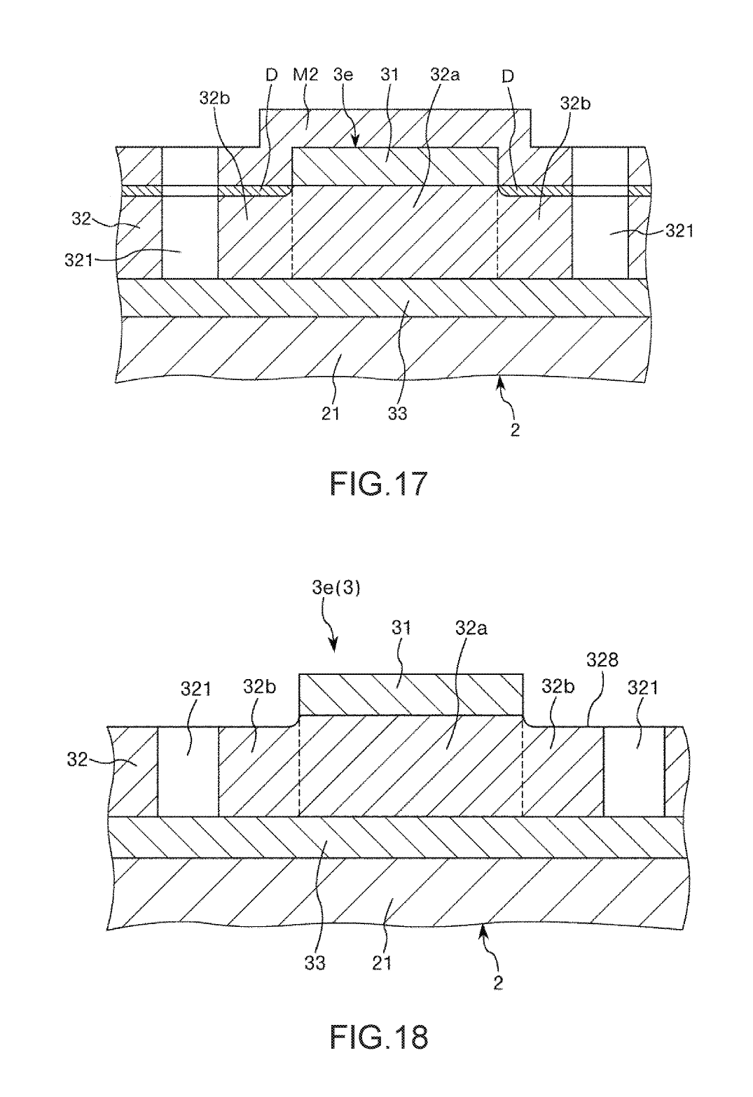

Next, the mask M2 (that is, resist mask) having an opening corresponding to the contact hole 321 is formed on the piezoelectric body 32 and the first electrode 31. The mask M2 can be formed, for example, in the same manner as the mask M2. Next, as illustrated in FIG. 17, the piezoelectric body 32 is dry-etched via the mask M2, and the piezoelectric body 32 is patterned. As a result, the contact hole 321 formed in the nonactive portion 32b of the piezoelectric body 32 is obtained. The process is performed before the light etching process, so that formation of a new damaged layer D after the light etching process can be suppressed. Next, the mask M2 is removed by asking treatment.

### Light Etching Process

Next, the piezoelectric body 32 is light-etched to remove the damaged layer D from the piezoelectric body 32 as illustrated in FIG. 18. As a result, the top surface 328 of the nonactive portion 32b can be configured to include the crystal surface which is substantially not damaged. The damaged layer D is removed by light etching, so that it is possible to effectively remove the damaged layer D while reducing damage to the piezoelectric body 32 (while suppressing the formation of a new damaged layer D).

Accordingly, the piezoelectric element 3 in which the damaged layer D is removed, the occurrence of cracks, burnout, and the like can be suppressed, the mechanical strength is improved, and the failure probability can be reduced can be obtained.

In the manufacturing method described above, although patterning is performed by dry etching in the first electrode patterning process and the piezoelectric body patterning process, the method of patterning in these processes is not limited to dry etching, and ion milling or wet etching may be used. Even in a case where the ion milling or the wet etching is used, similar to the case where the dry etching is used, the piezoelectric element 3 in which the mechanical strength is improved, and the failure probability can be reduced can be obtained.

### Second Embodiment

Next, a robot according to a second embodiment of the invention will be described.

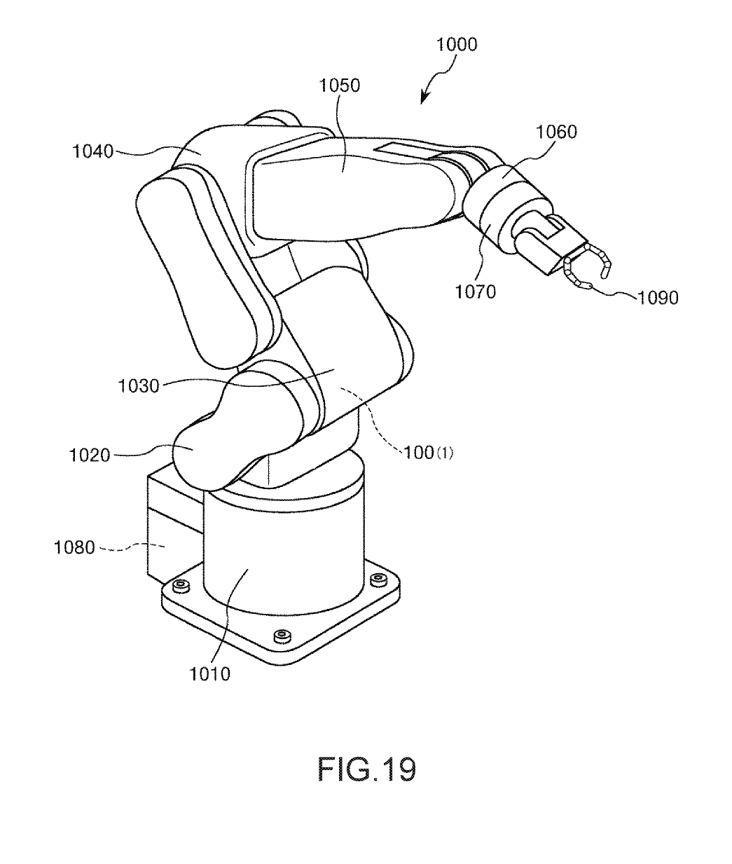

FIG. 19 is a perspective view illustrating a robot of a second embodiment of the invention.

The robot 1000 illustrated in FIG. 19 can perform work, such as supply, remove, transport, and assembly, of precision equipment or components (target objects) that configure the equipment. The robot 1000 is a 6-axis robot, and includes a base 1010 fixed to a floor or a ceiling, an arm 1020 which is linked to the base 1010 to be freely rotatable, an arm 1030 which is linked to the arm 1020 to be freely rotatable, an arm 1040 which is linked to the arm 1030 to be freely rotatable, an arm 1050 which is linked to the arm 1040 to be freely rotatable, an arm 1060 which is linked to the arm 1050 to be freely rotatable, an arm 1070 which is linked to the arm 1060 to be freely rotatable, and a control portion 1080 which controls the driving of the arms 1020, 1030, 1040, 1050, 1060, and 1070. In addition, a hand connection portion is provided in the arm 1070, and an end effector 1090 that corresponds to the work of executing the robot 1000 is mounted on the hand connection portion. In addition, the piezoelectric motor 100 (piezoelectric actuator 1) is mounted on the entire or a part of each of joint portions, and each of the arms 1020, 1030, 1040, 1050, 1060, and 1070 rotate by the driving of the piezoelectric motor 100. The driving of each of the piezoelectric motors 100 is controlled by the control portion 1080.

Such a robot 1000 is provided with the piezoelectric motor 100 (piezoelectric actuator 1 (piezoelectric element 3)). Therefore, the effect of the piezoelectric actuator 1 (piezoelectric element 3) described above is enjoyed and excellent reliability can be exhibited.

### Third Embodiment

Next, an electronic component transporting apparatus according to a third embodiment of the invention will be described.

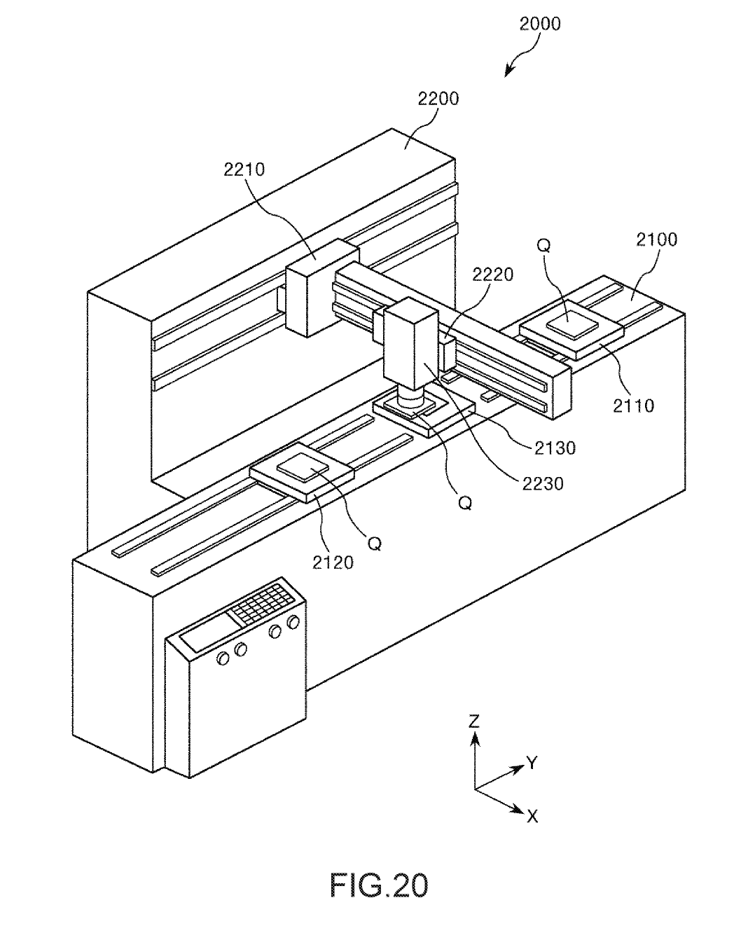

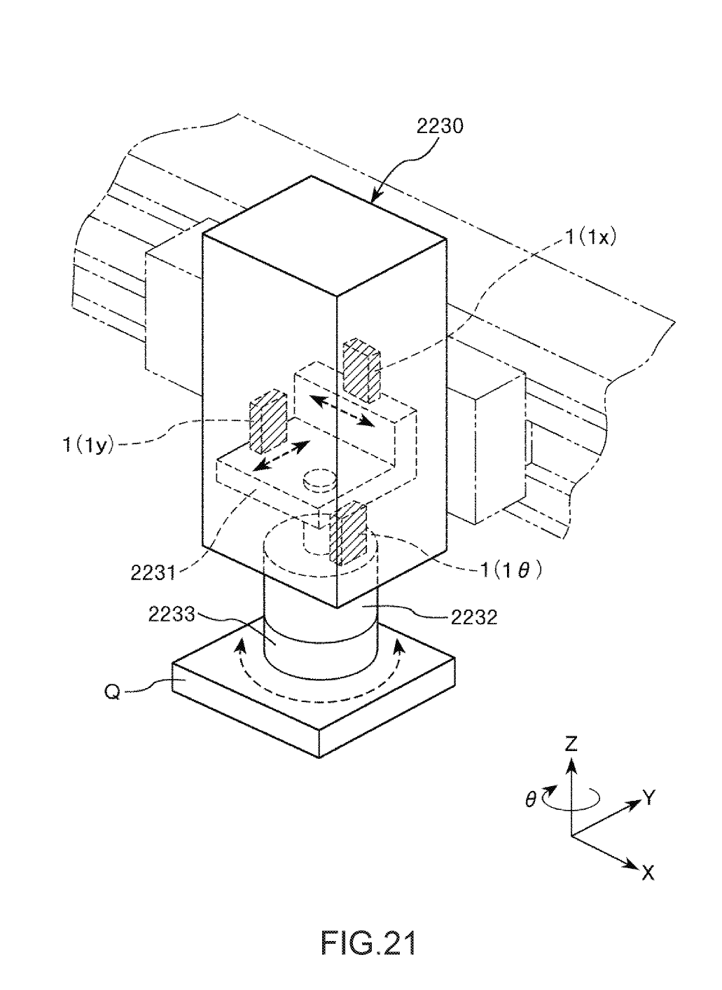

FIG. 20 is a perspective view illustrating an electronic component transporting apparatus of a third embodiment of the invention. FIG. 21 is a perspective view illustrating an electronic component holding portion included in the electronic component transporting apparatus illustrated in FIG. 20. Hereinafter, for the convenience of the description, three axes orthogonal to each other are set as X axis, Y axis, and Z axis.

An electronic component transporting apparatus 2000 illustrated in FIG. 20 is employed in an electronic component inspection apparatus, and includes a base table 2100, and a support table 2200 disposed on a side of the base table 2100. In addition, on the base table 2100, an upstream side stage 2110 on which an electronic component Q which is an inspection target is mounted and transported in a Y-axis direction, a downstream side stage 2120 on which the electronic component Q which is already inspected is mounted and transported in the Y-axis direction, and an inspection table 2130 which is positioned between the upstream side stage 2110 and the downstream side stage 2120 and inspects electric characteristics of the electronic component Q are provided. Examples of the electronic component Q include a semiconductor, a semiconductor wafer, a display device, such as CLD or OLED, a crystal device, various sensors, ink jet head, or various MEMS devices.

In addition, an Y stage 2210 which can move in the Y-axis direction with respect to the support table 2200 is provided on the support table 2200, an X stage 2220 which can move in an X-axis direction with respect to the Y stage 2210 is provided on the Y stage 2210, and an electronic component holding portion 2230 which can move in a Z-axis direction with respect to the X stage 2220 is provided on the X stage 2220. In addition, as illustrated in FIG. 21, the electronic component holding portion 2230 includes a fine adjustment plate 2231 which can move in the X-axis direction and in the Y-axis direction, a rotation portion 2232 which can rotate around a Z axis with respect to the fine adjustment plate 2231, and a holding portion 2233 which is provided in the rotation portion 2232 and holds the electronic component Q. In addition, in the electronic component holding portion 2230, the piezoelectric actuator 1 (1x) for moving the fine adjustment plate 2231 in the X-axis direction, the piezoelectric actuator 1 (1y) for moving the fine adjustment plate 2231 in the Y-axis direction, and the piezoelectric actuator 1 (1θ) for moving the rotation portion 2232 around the Z axis, are embedded.

Such an electronic component transporting apparatus 2000 is provided with the piezoelectric actuator 1 (piezoelectric element 3). Therefore, the effect of the piezoelectric element 3 described above is enjoyed and excellent reliability can be exhibited.

### Fourth Embodiment

Next, a printer according to a fourth embodiment of the invention will be described.

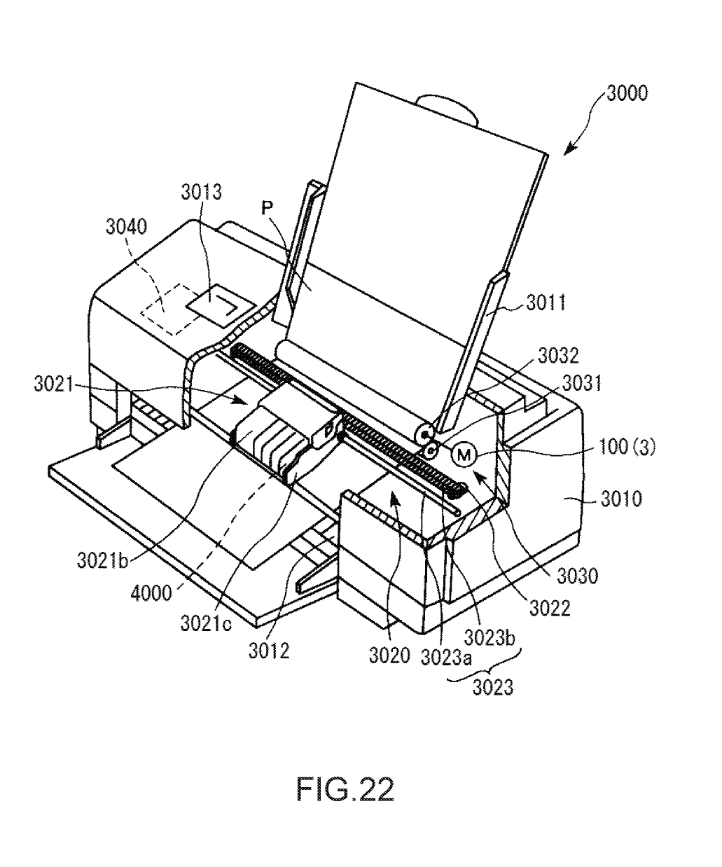

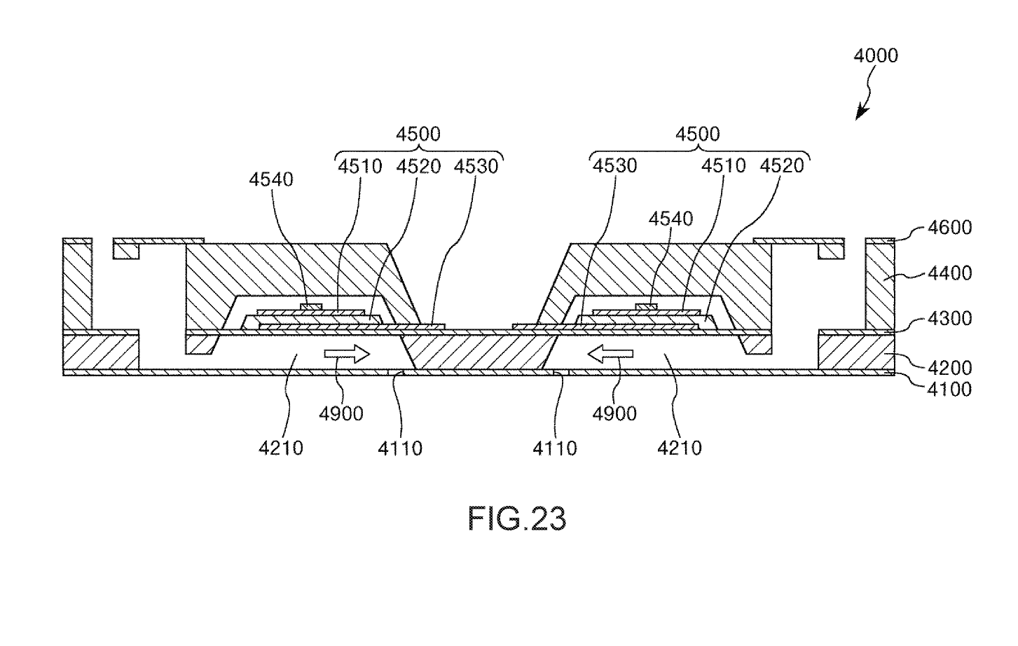

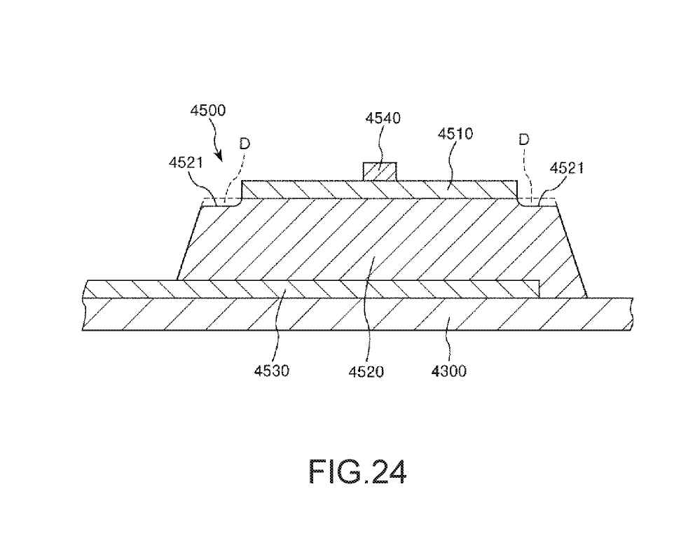

FIG. 22 is a schematic diagram illustrating an entire configuration of a printer according to a fourth embodiment of the invention. FIG. 23 is a sectional view of a head included in the printer illustrated in FIG. 22. FIG. 24 is a sectional view of a piezoelectric element included in the head illustrated in FIG. 23.

The printer 3000 illustrated in FIG. 22 includes an apparatus main body 3010, a printing mechanism 3020 provided on the inside of the apparatus main body 3010, a paper supply mechanism 3030, and a control portion 3040. In the apparatus main body 3010, a tray 3011 on which a recording paper sheet P is installed, a paper discharge port 3012 which discharges the recording paper sheet P, and an operation panel 3013 such as a liquid crystal display, are provided.

The printing mechanism 3020 includes a head unit 3021, a carriage motor 3022, and a reciprocating mechanism 3023 which reciprocates the head unit 3021 by the driving force of the carriage motor 3022. The head unit 3021 includes a head 4000 (liquid droplet ejecting head) which is an ink jet type recording head, an ink cartridge 3021b which supplies ink to the head 4000, and a carriage 3021c on which the head 4000 and the ink cartridge 3021b are mounted.

The reciprocating mechanism 3023 includes a carriage guide shaft 3023a which supports the carriage 3021c to be capable of reciprocating, and a timing belt 3023b which moves the carriage 3021c on the carriage guide shaft 3023a by the driving force of the carriage motor 3022. The paper supply mechanism 3030 includes a driven roller 3031 and a driving roller 3032 which are pressure-welded to each other, and the piezoelectric motor 100 which is a paper supply motor that drives the driving roller 3032. The control portion 3040 controls the printing mechanism 3020 or the paper supply mechanism 3030 based on printing data input from a host computer such as a personal computer.

In such a printer 3000, the paper supply mechanism 3030 intermittently feeds the recording paper sheet P in the vicinity of a lower portion of the head unit 3021 one by one. At this time, the head unit 3021 reciprocates in a direction which is substantially orthogonal to a feeding direction of the recording paper sheet P, and the printing onto the recording paper sheet P is performed.

Next, the head 4000 will be described in detail. As illustrated in FIG. 23, the head 4000 has a nozzle substrate 4100, a flow path forming substrate 4200, a diaphragm 4300, a reservoir forming substrate 4400, and a compliance substrate 4600. In addition, the nozzle substrate 4100, the flow path forming substrate 4200, the diaphragm 4300, the reservoir forming substrate 4400, and the compliance substrate 4600 are stacked in this order from the lower side in the drawing. In addition, in these substrates, two adjacent substrates are bonded to each other via, for example, an adhesive, a heat welding film or the like.

In addition, the head 4000 is disposed on the diaphragm 4300 and has a plurality of the piezoelectric elements 4500 covered with the reservoir forming substrate 4400. In such a head 4000, the piezoelectric element 4500 vibrates the diaphragm 4300, thereby changing the pressure inside a pressure generating chamber 4210 formed in the flow path forming substrate 4200, and an ejection port 4110 formed in the nozzle substrate 4100 is configured to eject ink 4900 as liquid droplets. The pressure generating chamber 4210 is disposed in two rows in the lateral direction of FIG. 23, and disposed in n rows (n is an integer of 1 or more) in the depth direction of FIG. 23.

In addition, the plurality of the piezoelectric elements 4500 have a second electrode 4530 disposed on the diaphragm 4300, a piezoelectric body 4520 disposed on the second electrode 4530, and a first electrode 4510 disposed on the piezoelectric body 4520. In addition, the second electrode 4530 is drawn out to the outside of the reservoir forming substrate 4400 along the top surface of the diaphragm 4300. The first electrode 4510 is drawn out to the outside of the reservoir forming substrate 4400 via a lead wiring 4540.

In addition, as illustrated in FIG. 24, in the piezoelectric element 4500, a portion of the top surface of the piezoelectric body 4520 is exposed from the first electrode 4510, and an exposed region 4521 (region located at the vicinity of the first electrode 4510 in plan view) is configured to include a crystal surface of the piezoelectric body 4520. That is, the damaged layer D is removed from the top surface of the piezoelectric body 4520, similar to the piezoelectric element 3 of the first embodiment described above.

Such a printer 3000 is provided with the piezoelectric motor 100 and the piezoelectric element 3. Therefore, the effect of the piezoelectric element 3 described above is enjoyed and excellent reliability can be exhibited. In the embodiment, the piezoelectric motor 100 drives the driving roller 3032 for paper supplying, but in addition to this, for example, the timing belt 3023b may be driven.

### Fifth Embodiment

Next, an ultrasonic transducer according to a fifth embodiment of the invention will be described.

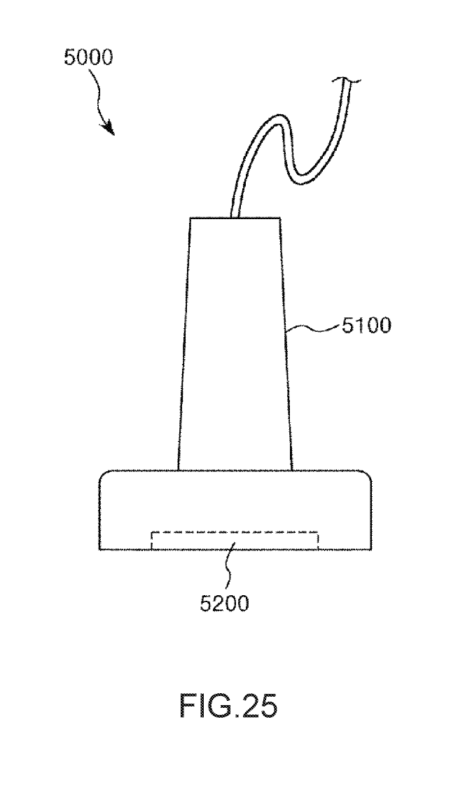

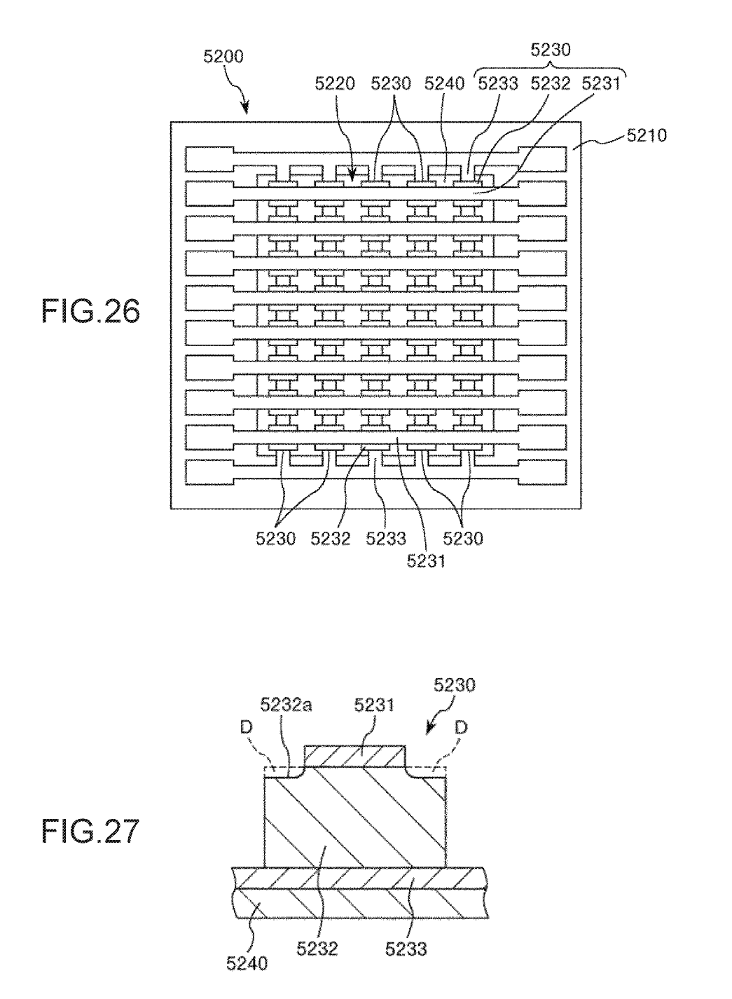

FIG. 25 is a schematic diagram illustrating an entire configuration of an ultrasonic transducer according to a fifth embodiment of the invention. FIG. 26 is a plan view of an element chip included in the ultrasonic transducer illustrated in FIG. 25. FIG. 27 is a sectional view of a piezoelectric element included in the element chip illustrated in FIG. 26.

As illustrated in FIG. 25, an ultrasonic wave probe 5000 as the ultrasonic transducer has a housing 5100 and the element chip 5200 which is disposed in the housing 5100 and whose surface is exposed on the surface of the housing 5100. The element chip 5200 can output ultrasonic waves from the surface and receive reflected waves of ultrasonic waves.

As illustrated in FIG. 26, the element chip 5200 has a substrate 5210, a flexible film 5240 supported on the substrate 5210, and an element array 5220 disposed on the flexible film 5240. In addition, the element array 5220 has a plurality of the piezoelectric elements 5230 disposed in a matrix.

In addition, each of the plurality of piezoelectric elements 5230 has a second electrode 5233 disposed on the flexible film 5240, a piezoelectric body 5232 disposed on the second electrode 5233, and a first electrode 5231 disposed on the top surface of the piezoelectric body 5232. The second electrode 5233 is a common electrode commonly provided for all the piezoelectric elements 5230. On the other hand, the first electrode 5231 is provided in common to the plurality of the piezoelectric elements 5230 aligned in the column direction (lateral direction). In such an element chip 5200, vibrations of the piezoelectric element 5230 causes the flexible film 5240 to vibrate, so that ultrasonic waves can be output, and conversely, vibration of the flexible film 5240 due to reflection of ultrasonic waves causes the piezoelectric element 5230 to be deformed, so that a detection signal can be obtained.

In addition, as illustrated in FIG. 27, in the piezoelectric element 5230, a portion of the top surface of the piezoelectric body 5232 is exposed from the first electrode 5231, the exposed region 5232a (region located at the vicinity of the first electrode 5231 in plan view) is configured to include the crystal surface of the piezoelectric body 5232. That is, the damaged layer D has been removed from the top surface of the piezoelectric body 5232, similar to the piezoelectric element 3 of the first embodiment described above.

In this manner, the ultrasonic wave probe 5000 as an example of the ultrasonic transducer is provided with the piezoelectric element 5230. Therefore, the effect of the piezoelectric element 5230 (the same effect as the piezoelectric element 3 described above) is enjoyed and excellent reliability can be exhibited.

Hereinbefore, the piezoelectric element, the piezoelectric actuator, the piezoelectric motor, the robot, the electronic component transporting apparatus, the printer, the ultrasonic transducer, and the method of manufacturing the piezoelectric element according to the invention are described based on the embodiments of the drawings, but the invention is not limited thereto, and the configuration of each portion can be changed to an arbitrary configuration having a similar function. In addition, other arbitrary configuration objects may be added to the invention. In addition, each of the embodiments may be appropriately combined with each other.

In addition, in the embodiment described above, the configuration in which the piezoelectric element is applied to the piezoelectric actuator, the piezoelectric motor, the robot, the electronic component transporting apparatus, the printer, and the ultrasonic transducer has been described, but the piezoelectric element can be applied to various electronic devices other than these.

The entire disclosure of Japanese Patent Application No. 2016-190782, filed Sep. 29, 2016 is expressly incorporated by reference herein.

What is claimed is:

1. A piezoelectric element comprising:
a piezoelectric body; and
a first electrode which is disposed on the piezoelectric body;
wherein in a plan view viewed from a direction where the first electrode and the piezoelectric body are aligned, a region which is a surface of the piezoelectric body on which the first electrode is disposed, located at a vicinity of the first electrode, and within 10 μm from an outer edge of the first electrode has a crystal surface.

2. The piezoelectric element according to claim 1, further comprising:
a second electrode which is disposed on the surface of the piezoelectric body opposite to the surface on which the first electrode is disposed,
wherein in the plan view viewed from the alignment direction, the second electrode has a portion overlapping with the crystal surface.

3. The piezoelectric element according to claim 1,
wherein in the plan view viewed from the alignment direction, when a portion overlapping with the first electrode of the piezoelectric body is set as a first portion and a portion located at the vicinity of the first electrode is set as a second portion, a length along the alignment direction of the second portion is shorter than a length along the alignment direction of the first portions.

4. The piezoelectric element according to claim 1,
wherein the piezoelectric body vibrates in a direction intersecting with the alignment direction.

5. A piezoelectric actuator comprising:
the piezoelectric element according to claim 1.

6. A piezoelectric actuator comprising:
the piezoelectric element according to claim 2.

7. A piezoelectric actuator comprising:
the piezoelectric element according to claim 3.

8. A piezoelectric motor comprising:
the piezoelectric actuator according to claim 5.

9. A robot comprising:
the piezoelectric element according to claim 1.

10. A robot comprising:
the piezoelectric element according to claim 2.

11. An electronic component transporting apparatus comprising:
the piezoelectric element according to claim 1.

12. An electronic component transporting apparatus comprising:
the piezoelectric element according to claim 2.

13. A printer comprising:
the piezoelectric element according to claim 1.

14. A printer comprising:
the piezoelectric element according to claim 2.

15. An ultrasonic transducer comprising:
the piezoelectric element according to claim 1.

16. An ultrasonic transducer comprising:
the piezoelectric element according to claim 2.

17. A method of manufacturing a piezoelectric element comprising:
preparing a piezoelectric body on which a metal film is disposed;
removing a portion of the metal film by dry etching or ion milling and patterning the metal film; and
light etching a portion of the piezoelectric body which is exposed from the metal film.

18. The method of manufacturing the piezoelectric element according to claim 17, further comprising:
forming a mask on the metal film, which is performed before the patterning of the metal film, and
removing the mask, which is performed after the patterning of the metal film and before the light etching.

19. The method of manufacturing the piezoelectric element according to claim 17, further comprising:
removing a portion of the piezoelectric body by dry etching or ion milling and patterning the piezoelectric body, which are performed after the patterning of the metal film and before the light etching.

20. The method of manufacturing the piezoelectric element according to claim 17,
wherein the piezoelectric body is formed using a solution method in the preparing.
